(19) United States
(12) Patent Application Publication
(10) Pub. No.: US 2009/0104594 A1
(43) Pub. Date: Apr. 23, 2009

(54) Bioreactor Process Control System and Method

(75) Inventor: Marcus Webb, Poway, CA (US)

Correspondence Address: STERNE, KESSLER, GOLDSTEIN & FOX, P.L.L.C., 1100 NEW YORK AVE., N.W., WASHINGTON, DC, 20005, US

(73) Assignee: Biogen Idec

(21) Appl. No.: 11/813,178

(22) PCT Filed: Dec. 22, 2005
(86) PCT No.: PCT/US2005/046545

§ 371 Date: May 15, 2008

Related U.S. Application Data
(60) Provisional application No. 60/639,816, filed on Dec. 29, 2004.

(51) Int. Cl. C12Q 3/00; C12M 1/34; C12M 1/36; C12M 1/38

(52) U.S. Cl. 435/3; 435/286.1; 435/286.6; 435/286.7

(57) ABSTRACT

A bioreactor includes a sensor linked to a model free adaptive controller or optimizer. The sensor can provide a real time measurement of a quantity that correlates with final product titer or other desired product quality attribute.

### CLAIM OF PRIORITY

This application claims priority to Provisional U.S. Application No. 60/639,816, filed Dec. 29, 2004, which is incorporated by reference in its entirety.

### TECHNICAL FIELD

This invention relates to a control system.

### BACKGROUND

Bioreactor control schemes use a number of individual single-input single-output (SISO) control loops to control variable such as temperature, agitation speed, pressure, dissolved oxygen, pH, etc., to specific setpoints. All the variables interact to varying degrees (in other words, their control loops are coupled) and have an effect on final product titer and other desired product quality attributes. The coupling between the control loops is generally ignored, and variable setpoints are fixed with the goal of consistently producing a given product and yield. Regulatory constraints have also reinforced this traditional method of SISO control methodologies for bioreactors, filings are made with the FDA that state the control schemes and associated setpoints of the control loops and after approval change is typically difficult to affect due to the regulated and highly controlled operating environment within FDA approved manufacturing facilities.

Typical advanced control strategies require a model of the process to be controlled. The model, however, is often difficult to determine and accurately validate. Furthermore, the model may change in real time, depending on the phase of the operation.

### SUMMARY

A bioreactor can be controlled using an adaptive controller. The adaptive controller can also be used to optimize bioreactor conditions. The adaptive controller can be, for example, a model-free adaptive controller (MFA). A model-free adaptive controller does not require a model of the process to be controlled. The input variables can be decoupled from one another and individually manipulated. The MFA controller can determine and actuate the required output variable changes to meet a desired input measurement. The input measurement can provide a real-time determination of a variable that correlates with final product titer (such as viable cell density (VCD)), or other desired product quality attribute or process indicator. Examples of suitable input measurements include carbon dioxide production rate, biomass concentration, oxygen uptake rate, substrate concentration, and glucose uptake rate. For example, the input measurement can be provided by a sensor monitoring a specific quality parameter in the bioreactor.

In one aspect, a bioreactor includes a cell growth vessel and a sensor, where the sensor is configured to measure a condition inside the vessel and provide an input to a model-free adaptive controller.

The sensor can measure a condition that correlates with a product quality attribute. The product quality attribute can be final product titer. The sensor can be configured to provide the input in real time. The sensor can measure viable cell density directly or indirectly. The model-free adaptive controller can be configured to compare the input to a setpoint. The model-free adaptive controller can be configured to provide an output to an actuator. The sensor can be configured to measure viable cell density, temperature, agitation speed, pressure, dissolved oxygen, or pH. The bioreactor can include a second sensor configured to measure a second condition inside the vessel and provide a second input to the model-free adaptive controller.

In another aspect, a method of culturing living cells includes incubating the cells in a vessel, measuring a condition inside the vessel, comparing the measurement to a setpoint with a model-free adaptive controller or optimizer, and adjusting a condition inside the vessel based on the comparison.

In another aspect, a method of culturing living cells includes incubating the cells in a vessel, measuring a plurality of conditions inside the vessel, comparing the plurality of measurements, individually, to a plurality of setpoints with a model-free adaptive controller, and adjusting a condition inside the vessel based on at least one comparison.

The condition can be viable cell density, temperature, agitation speed, dissolved oxygen, pH, turbidity, conductivity, pressure, NO/NOx, TOCNVOC, chlorine, ozone, oxidation-reduction potential, suspended solids, or another process condition measurement accomplished through other methods, such as, for example, electrochemical, infrared, optical chemical, radar, vision, radiation, pulse dispersion and mass spectrometry, acoustics, tomography, gas chromatography, liquid chromatography, spectrophotometry, opacity, thermal conductivity, refractometry, strain, or viscosity. A plurality of conditions inside the vessel can be adjusted based on at least one comparison. The condition can correlate with a product quality attribute. The product quality attribute can be final product titer. Measuring a condition can include measuring in real time. Measuring a condition can include measuring the viable cell density. The method can include adjusting the setpoint. The setpoint can be adjusted according to a predetermined trajectory. The trajectory can be optimized for a certain product quality attribute or multiple attributes.

In another aspect, a bioreactor includes a cell growth vessel, a sensor configured to measure a condition inside the vessel, wherein the condition correlates with final product titer, and a model-free adaptive controller configured to receive a measurement from the sensor and provide an output to an actuator.

The sensor can be configured to measure viable cell density. The sensor can be configured to measure the condition in real time.

In another aspect, a method of selecting conditions for a bioreactor process includes incubating a plurality of cells in a vessel, measuring a plurality of conditions inside the vessel, and determining a preferred level of a selected condition with a model-free adaptive controller. Determining a preferred level of a selected condition can include determining an optimum level of the condition.

The details of one or more embodiments are set forth in the accompanying drawings and the description below. Other features, objects, and advantages will be apparent from the description and drawings, and from the claims.

### DESCRIPTION OF DRAWINGS

FIG. 1 is a schematic depiction of a bioreactor.

FIG. 2 is a schematic depiction of a single input single output control loop.

FIGS. 3A-3D are graphs depicting desired trajectories and measured performance of a bioreactor process.

### DETAILED DESCRIPTION

In general, a bioreactor is a device for culturing living cells. The cells can produce a desired product, such as, for example, a protein, or a metabolite. The protein can be, for example a therapeutic protein, for example a protein that recognizes a desired target. The protein can be an antibody. The metabolite can be a substance produced by metabolic action of the cells, for example, a small molecule. A small molecule can have a molecular weight of less than 5,000 Da, or less than 1,000 Da. The metabolite can be, for example, a mono- or poly-saccharide, a lipid, a nucleic acid or nucleotide, a peptide (e.g., a small protein), a toxin, or an antibiotic.

The bioreactor can be, for example, a stirred-tank bioreactor. The bioreactor can include a tank holding a liquid medium in which living cells are suspended. The tank can include ports for adding or removing medium, adding gas or liquid to the tank (for example, to supply air to the tank, or adjust the pH of the medium with an acidic or basic solution), and ports that allow sensors to sample the space inside the tank. The sensors can measure conditions inside the bioreactor, such as, for example, temperature, pH, or dissolved oxygen concentration. The ports can be configured to maintain sterile conditions within the tank. Other bioreactor designs are known in the art. The bioreactor can be used for culturing eukaryotic cells, such as a yeast, insect, plant or animal cells; or for culturing prokaryotic cells, such as bacteria. Animal cells can include mammalian cells, an example of which is chinese hamster ovary (CHO) cells. In some circumstances, the bioreactor can have a support for cell attachment, for example when the cells to be cultured grow best when attached to a support. The tank can have a wide range of volume capacity—from 1 L or less to 10,000 L or more.

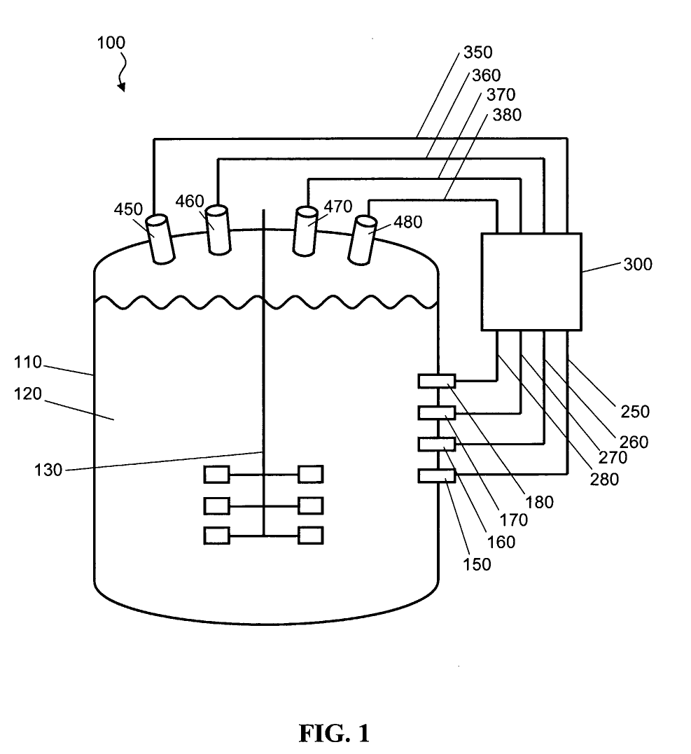

Referring to FIG. 1, bioreactor system 100 includes vessel 110 holding liquid cell culture 120 which can be stirred by agitator 130. Conditions inside the vessel are monitored by a plurality of sensors, shown as sensors 150, 160, 170 and 180. Each sensor independently provides a measurement as an input 250, 260, 270 and 280, respectively, to controller 300. Controller 300 compares each input to a setpoint and provides individual outputs 350, 360, 370 and 380. Each output 350, 360, 370 and 380 affects the operation of actuators 450, 460, 470 and 480, respectively. Operation of each of actuators 450, 460, 470 and 480, in turn, affects the conditions monitored by sensors 150, 160, 170 and 180, respectively. In this way, the control system of sensors, inputs, controller, outputs and actuators serves to maintain the monitored conditions inside the vessel at their setpoints. For reasons of clarity, bioreactor system 100 is illustrated with four groups of sensors, actuators, and associated inputs and outputs, but any number can be used. Sensors can be in contact with the liquid medium or with a headspace gas. The actuators can deliver material to the vessel (for example, an acidic or basic solution, to change the pH of the liquid medium) or can alter other functions of the bioreactor system (such as heating or agitation speed).

An important goal of bioreactor process control is to maximize the amount of product recovered at the end of the process (i.e., final product titer). A bioreactor is often controlled by fixing setpoints for each process parameter. The setpoints can remain fixed during one or more phases of the process or for the duration of the process. The setpoints can be determined ahead of time, for example in small-scale developmental tests of the process. In small scale tests, bioreactor conditions can be varied one at a time and an optimum level for each condition determined. These optimum levels can become the setpoints in large-scale process operations. However, the selected setpoints may not represent the best possible set of conditions for maximizing final product titer, for example, when a process is transferred to a large scale manufacturing environment or different process vessel configuration. Furthermore, product yield can vary from batch to batch, even when the bioreactor control conditions are identical for each batch. Batch-to-batch variability can be due to external inputs to the system such as raw materials. A component of a raw material may have a detrimental effect on the final product quality attribute of interest. A SISO control scheme that does not provide a real-time measure of the quality attribute of interest or the ability to influence multiple outputs and therefore can have no way of making the necessary corrective actions to account for the raw material variance.

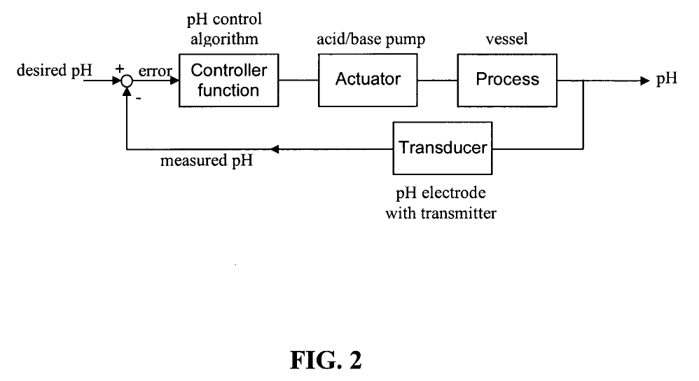

FIG. 2 represents a SISO control loop, using pH control as an example. In FIG. 2, pH is the variable subject to control by the pH control algorithm. The difference between the desired pH (i.e., the setpoint) and the measured pH is calculated to provide an error. The error is an input to the controller function, which provides an output to the actuator. For pH control, the actuator can be a pump that adds acid or base (as appropriate) to the vessel. The action of the actuator on the process (i.e., the conditions in the vessel) alters the pH, which is measured by a transducer (such as a pH electrode). Comparison of the measurement to the setpoint, and generation of the error signal again, completes the control loop.

Controller 300 can be an adaptive controller or optimizer, which can respond to changes in the process state by altering the setpoints of one or more process parameters. Using an adaptive controller to control aspects of a bioreactor process can improve product yield and the batch-to-batch reproducibility of product yield.

The adaptive controller can accept a real-time input. The real-time input can be a measurement of a process parameter. The adaptive controller can respond to changes in the real-time input by altering a setpoint of a process parameter. The real-time input can be a measurement that correlates with final product titer.

Adaptive controllers frequently require a model of the process to work. The model can include information about the coupling of control loops: how changes in one process parameter affect other process parameters. For example, a change in temperature might result in a change in pH. The model used in the adaptive controller must accurately reflect the couplings between all control loops in order to successfully control the process. An accurate model can be difficult or impossible to determine. Even when a model is used successfully, it may only be effective when the process parameters are close to the respective setpoints around which the model is observed and constructed.

The adaptive controller can be a model-free adaptive (MFA) controller. The adaptive controller can be used as an optimizer, i.e., to identify preferred conditions for the process. A model-free adaptive controller is a controller that can alter setpoints of process conditions, but does not use a mathematical model of the process. The MFA controller uses a dynamic feedback system to adjust the output and setpoint. The dynamic feedback system can be an artificial neural network. The MFA controller can be a single input single output (SISO) controller or a multiple input multiple output (MIMO) controller. MFA controllers are described in, for example, U.S. Pat. Nos. 6,055,524; 6,360,131; 6,556,980; 6,684,112; and 6,684,115; each of which is incorporated by reference in its entirety.

Unlike other adaptive controllers, a MFA controller does not require a model of the process to be controlled. Because the MFA controller does not use a model, it can be employed for processes for which no model can be determined, or operate successfully under conditions where the model does not accurately describe the process. The MFA controller can be appropriate for processes with coupled control loops where the coupling between the control loops is not fully understood. Frequently, bioreactor processes have coupled control loops and cannot be modeled accurately.

Measurements of product titer are often performed off-line and are not available until some time has elapsed. The delay between starting a product titer measurement (e.g., by collecting a sample from the bioreactor) and completing the measurement is often so long the information cannot be used for real-time bioreactor control purposes. A real-time sensor that provides information about the product titer, or other product quality attribute of interest, can be used as an input to the controller. The controller can adjust the output or setpoint of one or more process variables in order to keep the product titer at its setpoint.

A setpoint trajectory can be defined for a variable. The variable can be the product titer or other product quality attribute of interest. The setpoint trajectory can be optimized to maximize the product quality attribute of interest, or the setpoint trajectory can be optimized to maintain a desired specification for the product quality attribute. The setpoint can change as a function of time during the process. For a bioreactor process, a trajectory for viable cell density can be chosen, such as an ideal or theoretical growth curve for the cells. In this way the controller can drive the process along a consistent, reproducible path, even on different batches.

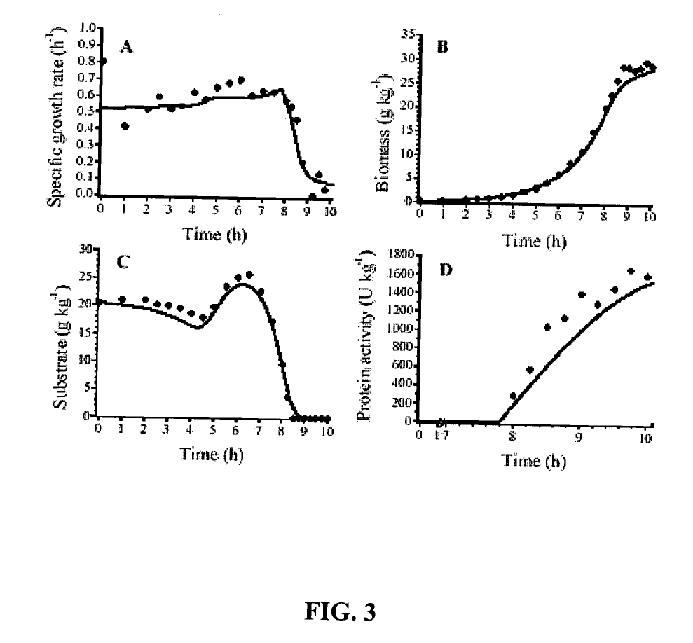

FIGS. 3A-3D are graphs showing exemplary trajectories for a bioreactor process. In each of FIGS. 3A-3D, the horizontal axis represents time. The solid lines represent the trajectories, and the circles represent real-time measurements for the process variables. The variables shown are specific growth rate (FIG. 3A), biomass (FIG. 3B), substrate concentration (FIG. 3C), and protein activity (FIG. 3D).

The final product titer can be influenced by the number of living cells present in the bioreactor. The number of living cells can follow a growth trajectory, or in other words, the number of living cells can increase as a function of time during the process according to a predetermined path. The path can include, for example, a lag phase, an exponential growth phase and a stationary phase. More particularly, the viable biomass present in the bioreactor can affect final product titer.

Sensors 150, 160, 170 and 180 can be real-time sensors, or delayed sensors. A real-time sensor provides measurements of the monitored condition as it occurs. A delayed sensor, in contrast, introduces a lag time between the moment the condition is measured and the moment the measurement is reported. For example, a delayed sensor can be an off-line sensor, where a sample of the liquid media must be removed from the vessel and transferred to another location for the measurement to occur.

Real-time sensors can be correlated with final product titer. For example, VCD can be measured by a capacitance-based sensor. Other parameters can be measured NIR-, Raman-, or fluorescence-based sensors. Because these measurements are taken in real time, they can be used for process control. Other real-time sensor measurement techniques include, for example, pH, temperature, turbidity, conductivity, pressure, electrochemical, infrared, optical chemical, radar, vision, radiation, pulse dispersion and mass spectrometry, acoustics, tomography, gas or liquid chromatography, spectrophotometers, multi-component and multi-sensor analyzers, opacity, oxygen, NO/NOx analyzers, thermal conductivity, TOC/VOC analyzers, chlorine, concentration, dissolved oxygen, ozone, ORP sensors, refractometer, suspended solids, strain gauges, nuclear, viscosity, x-ray, hydrogen.

Sensors and their use in control systems are described in, for example, Bentley, J. P. Principles of Measurement Systems; Liptak, B. G., Instrument Engineers Handbook, 3rd edition and Instrument Engineers Handbook, Volume 1, 4th Edition; Spitzer, D. W., Flow Measurement Practical Guides for Measurement & Control; and Perry R. H. and Green, D. W., Perry's Chemical Engineer's Handbook, each of which is incorporated by reference in its entirety. On-line and real-time sensors can be obtained from, for example, Emerson Process Management, ABB, Foxboro, Yokogawa, and Broadley-James.

Viable cell density (VCD) can be measured, for example, by obtaining a sample of culture medium and counting the number of cells present. Viable cell density can be measured with a radio-frequency impendence measurement. Cells with intact plasma membranes can act as tiny capacitors under the influence of an electric field. The non-conducting nature of the plasma membrane allows a buildup of charge. The resulting capacitance can be measured; it is dependent on the cell type and is proportional to the concentration of viable cells present. A four-electrode probe applies a low-current RF field to the biomass passing within 20 to 25 mm of the electrodes. The probe is insensitive to cells with leaky membranes, gas bubbles, cell debris, and other media components, so it detects only viable cells. Unlike optical probes, it is not prone to fouling, and provides a linear response over a wide range of viable cell concentrations. A system for measuring VCD in real time during a bioreactor process is available commercially, for example, from Aber Instruments, Aberystwyth, UK. See, for example, Carvell, J. P, Bioprocess International, January 2003, 2-7; and Ducommun, P. et al., Biotech. and Bioeng. (2002) 77, 316-323, each of which is incorporated by reference in its entirety.

The cells grown in a bioreactor can be engineered to produce a substance which is easily measured. The easily-measured substance preferably is one that is produced and/or removed at known or predictable rates, such that measuring the amount (or concentration) of substance in the media provides information about the cells. For example, the amount or concentration of the substance can be related to the cell number, biomass, or viable cell density. The easily-measured substance can be, for example, a light emitting substance. The substance is preferably measured by a real-time sensor.

For example, the cells can be engineered to express a fluorescent protein, such as a green fluorescent protein. The quantity of fluorescent protein expressed, and therefore the fluorescence intensity of the cell culture, can be related to the viable cell density. A sensor that measures the fluorescence intensity of a fluorescent protein can be incorporated into a bioreactor. See, for example, Randers-Eichhorn, L. et al., Biotech. and Bioeng. (1997) 55, 921-926, which is incorporated by reference in its entirety.

A sensor can monitor the presence of one or more compounds in the growth medium, for example by using IR or Raman spectroscopy. IR spectroscopy can be used, for example, to measure the concentration of gases such as NO, SO₂, CH₄, CO₂ and CO. Raman spectroscopy is the measurement of the wavelength and intensity of scattered light from molecules. However, a small fraction is scattered in other directions. Using Raman spectroscopy, the Raman probe can detect organic or inorganic compounds in the media surrounding the probe. The probe uses laser light beamed through a sapphire window. When the light hits the sample, it causes molecules to vibrate in a distinctive way, creating a fingerprint. The fingerprint is captured and transmitted via fiber optic cables to an analyzer, where it is compared to known signals.

The sensors can be used with a bioreactor that is controlled by a model-free adaptive controller or optimizer. The model free adaptive controller can receive an input from a real time sensor that correlates with final product titer. The sensor can be, for example, a capacitance sensor, a NIR sensor, a Raman sensor or a fluorescence sensor. The sensor can measure viable cell density, biomass, green fluorescent protein, or other desired product quality attribute, such as, for example, a substance in the medium. The substance can be, for example and without limitation, a fatty acid, a gas, an amino acid, or a sugar. The MFA controller can operate as a multiple input multiple output (MIMO) controller that adjusts several process variables. Any controlled process variable can be controlled by the MFA controller, such as, for example, temperature, pressure, pH, dissolved oxygen, or agitation speed. The MFA controller can be configured to maximize the final product titer.

The controller can provide outputs that control actuators, which in turn adjust the level of the process variables. Each process variable can have a setpoint. The inputs can be compared to the corresponding setpoints. Each output can be of a sign and magnitude to adjust the process variable towards its corresponding setpoint, reducing the difference between the input and the setpoint. The setpoint for each input can be adjusted by the controller.

For example, if during the process, the temperature inside the vessel falls below the setpoint, the controller can respond by sending an output to an actuator, such as a heater, that affects temperature. The output can be a positive output; i.e., it increase the activity of the heater so as to increase the temperature to the setpoint. The magnitude of the output can depend on the degree of error between the setpoint and the measured variable.

The setpoint adjustment can be designed to maximize a particular input. The maximized input can be an input that correlates with final product titer. The setpoints can be adjusted according to a predetermined trajectory, changing as a function of time, cell density, or other process variable, or other product quality attribute. The trajectory can be chosen to maximize final product titer.

A number of embodiments have been described. Nevertheless, it will be understood that various modifications may be made. Accordingly, other embodiments are within the scope of the following claims.

What is claimed is:

1. A bioreactor comprising a cell growth vessel and a sensor; wherein the sensor is configured to measure a condition inside the vessel and provide an input to a model-free adaptive controller.

2. The bioreactor of claim 1, wherein the sensor measures a condition that correlates with a product quality attribute.

3. The bioreactor of claim 2, wherein the product quality attribute is final product titer.

4. The bioreactor of claim 2, wherein the sensor is configured to provide the input in real time.

5. The bioreactor of claim 4, wherein the sensor measures viable cell density directly or indirectly.

6. The bioreactor of claim 1, wherein the model-free adaptive controller is configured to compare the input to a setpoint.

7. The bioreactor of claim 1, wherein the model-free adaptive controller is configured to provide an output to an actuator.

8. The bioreactor of claim 1, wherein the sensor is configured to measure viable cell density, temperature, agitation speed, dissolved oxygen, pH, turbidity, conductivity, pressure, NO/NOx, TOC/VOC, chlorine, ozone, oxidation-reduction potential, viscosity or suspended solids.

9. The bioreactor of claim 1, wherein the sensor is configured to measure the condition using a method selected from the group consisting of: electrochemical, infrared, optical chemical, radar, vision, radiation, pulse dispersion and mass spectrometry, acoustics, tomography, gas chromatography, liquid chromatography, spectrophotometry, opacity, thermal conductivity, refractometry, and strain.

10. The bioreactor of claim 1, further comprising a second sensor configured to measure a second condition inside the vessel and provide a second input to the model-free adaptive controller.

11. A method of culturing living cells comprising:
incubating the cells in a vessel;
measuring a condition inside the vessel;
comparing the measurement to a setpoint with a model-free adaptive controller; and
adjusting a condition inside the vessel based on the comparison.

12. The method of claim 11, wherein the condition is viable cell density, temperature, agitation speed, dissolved oxygen, pH, turbidity, conductivity, pressure, NO/NOx, TOC/VOC, chlorine, ozone, oxidation-reduction potential, viscosity or suspended solids.

13. The method of claim 11, wherein measuring a condition includes using a method selected from the group consisting of: electrochemical, infrared, optical chemical, radar, vision, radiation, pulse dispersion and mass spectrometry, acoustics, tomography, gas chromatography, liquid chromatography, spectrophotometry, opacity, thermal conductivity, refractometry, and strain.

14. The method of claim 11, wherein the condition is a condition that correlates with a product quality attribute.

15. The method of claim 14, wherein the product quality attribute is final product titer.

16. The method of claim 14, wherein measuring a condition includes measuring in real time.

17. The method of claim 15, wherein measuring a condition includes measuring the viable cell density.

18. The method of claim 11, further comprising adjusting the setpoint.

19. The method of claim 18, wherein the setpoint is adjusted according to a predetermined trajectory.

20. A method of culturing living cells comprising:
incubating the cells in a vessel;
measuring a plurality of conditions inside the vessel;
comparing the plurality of measurements, individually, to a plurality of setpoints with a model-free adaptive controller; and
adjusting a first condition inside the vessel based on at least one comparison.

21. The method of claim 20, wherein at least one measured condition is viable cell density, temperature, agitation speed, dissolved oxygen, pH, turbidity, conductivity, pressure, NO/NOx, TOCNVOC, chlorine, ozone, oxidation-reduction potential, viscosity or suspended solids.

22. The method of claim 20, wherein measuring a plurality of conditions includes using a method selected from the group consisting of: electrochemical, infrared, optical chemical, radar, vision, radiation, pulse dispersion and mass spectrometry, acoustics, tomography, gas chromatography, liquid chromatography, spectrophotometry, opacity, thermal conductivity, refractometry, and strain.

23. The method of claim 20, further comprising adjusting a plurality of conditions inside the vessel based on at least one comparison.

24. The method of claim 20, wherein at least one measured condition is a condition that correlates with a product quality attribute.

25. The method of claim 24, wherein the product quality attribute is final product titer.

26. The method of claim 24, wherein at least one measured condition is measured in real time.

27. The method of claim 26, wherein viable cell density is measured in real time.

28. The method of claim 20, further comprising adjusting at least one setpoint.

29. The method of claim 28, wherein the setpoint is adjusted according to a predetermined trajectory.

30. A bioreactor comprising:
a cell growth vessel;
a sensor configured to measure a condition inside the vessel, wherein the condition correlates with final product titer; and
a model-free adaptive controller configured to receive a measurement from the sensor and provide an output to an actuator.

31. The bioreactor of claim 30, wherein the sensor is configured to measure viable cell density.

32. The bioreactor of claim 30, wherein the sensor is configured to measure the condition in real time.

33. A method of selecting conditions for a bioreactor process comprising:
incubating a plurality of cells in a vessel;
measuring a plurality of conditions inside the vessel; and
determining a preferred level of a selected condition with a model-free adaptive controller.

34. The method of claim 33, wherein determining a preferred level of a selected condition includes determining an optimum level of the condition.
